(19) United States
(12) Patent Application Publication
(10) Pub. No.: US 2023/0249154 A1
(43) Pub. Date: Aug. 10, 2023

(54) ANTI-VIRAL GRANULAR ACTIVATED CARBON FOR GAS PHASE FILTRATION APPLICATIONS

(71) Applicant: CALGON CARBON CORPORATION, Moon Township, PA (US)

(72) Inventors: Jason P. MAZZOCCOLI, Pittsburgh, PA (US); John MATTHIS, Canonsburg, PA (US); Richard A. MIMNA, Oakdale, PA (US)

(73) Assignee: CALGON CARBON CORPORATION, Moon Township, PA (US)

(21) Appl. No.: 18/165,537

(22) Filed: Feb. 7, 2023

Related U.S. Application Data
(60) Provisional application No. 63/307,804, filed on Feb. 8, 2022.

(51) Int. Cl. B01J 20/20; A61L 9/16; B01J 20/28; B01J 20/02; B01J 20/22; B01D 53/04; B01D 46/00

(52) U.S. Cl. CPC B01J 20/20; A61L 9/16; B01J 20/28016; B01J 20/0251; B01J 20/0237; B01J 20/0244; B01J 20/22; B01D 53/04; B01D 46/0028; A61L 2209/22; B01D 2253/102; B01D 2253/25; B01D 2279/65; B01D 2253/1122

(57) ABSTRACT

The present application relates to activated carbon compositions for removing viral, bacterial, or other infectious particles from air. The activated carbon was found to be effective at filtering aerosolized bacteriophage particulates and could be applied to barrier materials which prevents viral particulates from passing through, for example in home filters or masks or other coverings to prevent the spread of infectious diseases.

### CROSS REFERENCE TO RELATED APPLICATIONS

This application claims priority to U.S. Provisional Application No. 63/307,804, filed on Feb. 8, 2022, which is hereby incorporated by reference in its entirety.

### FIELD

The present disclosure relates to activated carbon materials for use in filtering aerosolized particles. More specifically, the present disclosure relates to the use of impregnated activated carbon to filter viral particulates from air. This disclosure further relates to anti-viral activated carbon filtration products.

### BACKGROUND

Activated carbon is well-known for its ability to remove unwanted particulates from gas and fluid streams and is commonly used in the water purification industry. However, many technology fields benefit from the purification capabilities of activated carbon and other porous sorbent materials. The specific size and composition of sorbent materials allow their application in removing numerous types of contaminants from varied sources.

Viruses and bacteria are often transmitted person-to-person through aerosolized respiratory droplets that may inadvertently be inhaled or swallowed, or land on surfaces that can lead to contact transmission, or by smaller aerosolized particles that can linger in the air. Minimizing the potential for contact with these viral particles is crucial to reducing the spread of viruses, especially in the case of highly infectious strains.

The use of masks and other barriers are known to be effective in reducing the spread of infectious diseases. However, a physical barrier alone does not eliminate 100% of the viral particles that may reach the wearer. An additional filter material, along with a physical barrier, provide increased protection from viral particles and minimize the risk of a contagious individual further spreading the infection.

### SUMMARY

In some aspects, the techniques described herein relate to a filtration system for filtering viral particles from a gas stream, including: a first filter including activated carbon impregnated with an additive including iron, cobalt, cerium, nickel, copper, zinc, silver, a quaternary ammonium compound, or combinations thereof, and a second filter which does not include activated carbon.

In some aspects, the techniques described herein relate to a filtration system, wherein the activated carbon is formed from at least one of bagasse, bamboo, coconut husks, peat, wood such as hardwood and softwood sources in the form of sawdust and scrap, lignite, coal, coal tar, petroleum pitch, asphalt and bitumen, corn stalks and husks, wheat straw, spent grains, rice hulls and husks, nutshells, or combinations thereof.

In some aspects, the techniques described herein relate to a filtration system, wherein the activated carbon is granular.

In some aspects, the techniques described herein relate to a filtration system, wherein the additive includes elemental iron, iron oxide, iron oxyhydroxide, elemental copper, cuprous oxide, cupric oxide, elemental zinc, zinc oxide, elemental silver, silver oxide, silver nitrate, silver sulfate, silver phosphate, benzalkonium chloride, or combinations thereof.

In some aspects, the techniques described herein relate to a filtration system, wherein the activated carbon includes about 0.1 wt. % to about 10 wt. % of the additive.

In some aspects, the techniques described herein relate to a filtration system, wherein the activated carbon includes about 0.1 wt. % to about 2 wt. % of the additive.

In some aspects, the techniques described herein relate to a filtration system, wherein the activated carbon includes about 1 wt. % to about 5 wt. % of the additive.

In some aspects, the techniques described herein relate to a filtration system, wherein the activated carbon includes about 5 wt. % to about 10 wt. % of the additive.

In some aspects, the techniques described herein relate to a filtration system, wherein the second filter includes ceramic, porous membranes, fibers, glass, fiberglass, polypropylene, polyethylene terephthalate, cellulose, plastic, mesh, foam, sponge, high-efficiency particle air filter, or combinations thereof.

In some aspects, the techniques described herein relate to a filtration system, wherein the filtration system is contained within a housing, a wearable device, or combinations thereof.

In some aspects, the techniques described herein relate to a filtration system, wherein the contacting the filtration system with the gas stream results in at least about 95% reduction in viral particles.

In some aspects, the techniques described herein relate to a method of filtering viral particles from a gas stream that contains viral particles, including: contacting the gas stream with a filtration system including: a first filter including activated carbon impregnated with an additive including iron, copper, zinc, cerium, silver, a quaternary ammonium compound, or combinations thereof, and a second filter.

In some aspects, the techniques described herein relate to a method, wherein the activated carbon is formed from at least one of bagasse, bamboo, coconut husks, peat, wood such as hardwood and softwood sources in the form of sawdust and scrap, lignite, coal, coal tar, petroleum pitch, asphalt and bitumen, corn stalks and husks, wheat straw, spent grains, rice hulls and husks, nutshells, or combinations thereof.

In some aspects, the techniques described herein relate to a method, wherein the activated carbon is granular.

In some aspects, the techniques described herein relate to a method, wherein the activated carbon includes about 0.1 wt. % to about 10 wt. % of the additive.

In some aspects, the techniques described herein relate to a method, wherein the second filter does not include activated carbon.

In some aspects, the techniques described herein relate to a method, wherein the second filter includes ceramic, porous membranes, fibers, glass, fiberglass, polypropylene, polyethylene terephthalate, cellulose, plastic, mesh, foam, sponge, high-efficiency particle air filter, or combinations thereof.

In some aspects, the techniques described herein relate to a method, wherein the filtration system is contained within a housing, a wearable device, or combinations thereof.

In some aspects, the techniques described herein relate to a method, wherein contacting the gas stream with the filtration system results in at least about 95% reduction in viral particles.

In some aspects, the techniques described herein relate to a method, wherein contacting the gas stream with the filtration system results in at least about 98% reduction in viral particles.

### BRIEF DESCRIPTION OF DRAWINGS

Aspects, features, benefits and advantages of the embodiments described herein will be apparent with regard to the following description, appended claims, and accompanying drawings where:

FIG. 1 shows a flow diagram of the system used for testing the efficacy of the activated carbon materials.

FIG. 2 shows a particle size distribution graph of MS2 bacteriophage particles.

FIG. 3 shows the relative performance of six activated carbon compositions, each showing >1 LOG reduction for each composition.

FIG. 4 shows the relative performance of six activated carbon compositions, each showing >96% single pass reduction.

### DETAILED DESCRIPTION

Before the present compositions and methods are described, it is to be understood that the subject matter herein is not limited to the particular processes, compositions, or methodologies described, as these may vary. It is also to be understood that the terminology used in the description is for the purpose of describing the particular versions or embodiments only, and is not intended to limit the scope of the present subject matter, which will be limited only by the appended claims. Unless defined otherwise, all technical and scientific terms used herein have the same meaning as commonly understood by one of ordinary skill in the art. Although any methods and materials similar or equivalent to those described herein can be used in the practice or testing of embodiments of the present subject matter, the preferred methods, devices, and materials are now described. All publications mentioned herein are incorporated by reference in their entirety. Nothing herein is to be construed as an admission that the subject matter is not entitled to antedate such disclosure by virtue of prior invention.

It must also be noted that as used herein and in the appended claims, the singular forms “a,” “an,” and “the” include plural reference unless the context clearly dictates otherwise. Thus, for example, reference to “a combustion chamber” is a reference to “one or more combustion chambers” and equivalents thereof known to those skilled in the art, and so forth. Further, as used in this document, the term “comprising” means “including, but not limited to.”

As used herein, the term “about” means plus or minus 10% of the numerical value of the number with which it is being used. Therefore, “about 50” means in the range of 45-55.

As used herein, the term “sorbent material” is meant to encompass all known materials from any source. For example, sorbent materials include, but are not limited to, activated carbon, natural and synthetic zeolite, silica, silica gel, alumina, zirconia, and diatomaceous earths.

This disclosure describes filtration systems containing an activated carbon filter in combination with a non-activated carbon filter for filtering viral particles from a gas stream. The filtration system may be incorporated into any physical barrier or other filtration product known to one of skill in the art, including but not limited to face masks, face shields, air filters, air purifiers, or other filtration systems.

In some embodiments, there is provided a filtration system for removing viral particles from a gas stream. The filtration system may include a first filter which includes activated carbon impregnated with an additive which includes iron, cobalt, cerium, nickel, copper, zinc, silver, a quaternary ammonium compound, or combinations thereof, and a second filter which does not include activated carbon.

The term “activated carbon” is used to describe a range of similar sorbent materials that may be produced from any of a number of distinct starting materials. Activated carbon may be formed from materials including bagasse, bamboo, coconut husks, peat, wood such as hardwood and softwood sources in the form of sawdust and scrap, lignite, coal, coal tar, petroleum pitch, asphalt and bitumen, corn stalks and husks, wheat straw, spent grains, rice hulls and husks, nutshells, and combinations thereof.

Activated carbon is typically produced the thermal or chemical treatment of carbonaceous materials, and varying production methods may result in activated carbon having different size, surface characteristics, and other features, meaning that methods of production are often highly depending on end use of the product. In some embodiments, the activated carbon of the present disclosure is granular.

Activated carbon having an additive incorporated therein may be produced by impregnating the activated carbon with the additive. The step of impregnating is well known in the art and can be carried out in any number of ways. Typically, impregnating includes the step of contacting a sorbent material, by immersion or other means, with an impregnation solution containing one or more additives that are dissolved or dispersed in the impregnating solution. The impregnating solution may include one or more additives that will become associated with the sorbent material while the sorbent material is in contact with the impregnating solution. Impregnating can be carried out in one or more impregnating steps. For example, in some embodiments, all of the additives incorporated onto the sorbent material may be included in the impregnating solution such that all of the additives can become associated with the sorbent material in a single impregnating step. In other embodiments, the impregnating solution may include a single additive and a separate impregnating step may be necessary for each additive incorporated onto the sorbent material. In still other embodiments, impregnating can be carried out by impregnating with a first impregnating solution including two or more additives and impregnating with a second impregnating solution including one or more additives. In yet other embodiments, impregnating can be carried out using three or more impregnating steps in which each impregnating solution includes one, two, three, four, or more additives. In some embodiments, impregnating the sorbent material with an additive as described herein forms an impregnated sorbent material.

In some embodiments, the activated carbon of the present disclosure is impregnated with an additive. The additive may, in some embodiments, include iron, cobalt, cerium, nickel, copper, zinc, silver, a quaternary ammonium compound, or combinations thereof. In some embodiments, the additive is present in its elemental form, such as for example silver metal, and in other embodiments the additive is present in other forms, such as for example oxides, nitrates, sulfates, phosphates, or other compounds containing iron, cobalt, cerium, nickel, copper, zinc, or silver. In some embodiments, the additive includes elemental iron, iron oxide, iron oxyhydroxide, elemental copper, cuprous oxide, cupric oxide, elemental zinc, zinc oxide, elemental silver, silver oxide, silver nitrate, silver sulfate, silver phosphate, benzalkonium chloride, or combinations thereof.

In some embodiments, the activated carbon of the present disclosure includes an additive in the amount of about 0.1 wt. % to about 15 wt. %, for example, about 0.1 wt. %, about 0.2 wt. %, about 0.3 wt. %, about 0.4 wt. %, about 0.5 wt. %, about 0.6 wt. %, about 0.7 wt. %, about 0.8 wt. %, about 0.9 wt. %, about 1 wt. %, about 2 wt. %, about 3 wt. %, about 4 wt. %, about 5 wt. %, about 6 wt. %, about 7 wt. %, about 8 wt. %, about 9 wt. %, about 10 wt. %, about 11 wt. %, about 12 wt. %, about 13 wt. %, about 14 wt. %, about 15 wt. %, or any range or value contained therein.

In some embodiments, the filtration system is configured such that the first filter and the second filter are contained within a housing. The filters of various embodiments may have any design and may at least include a housing, including a compartment configured to hold the filter and allow streams of fluid to flow over or through the filter. Such filters may include various additional components such as, for example, screens or other means for holding the activated carbon in the compartment or additional purification devices such as filtration membranes, particulate filters, and the like. In some embodiments, the housing may include various components necessary to allow the filter to be integrated into a device such large-scale air purifiers in which fluid stream, such as air containing vehicle exhaust, flow from one compartment to another and pass through the filter during transfer. In particular, the filter may include an inlet port for introducing streams of fluid into the filter and an outlet port for dispensing the filtered streams of fluid from the filter. In some embodiments, the filter may include a removable connecting means to connect to a gas source such as a pipe, hose, tube fittings, and the like at the inlet port.

The second filter used in the filtration system of the present disclosure is not particularly limited, so long as it does not include activated carbon. In some embodiments, the second filter is any non-adsorbent filter known to those skilled in the art, such as electrostatic filters, mechanical filters, and the like. Examples of filters that may be used in embodiments of the present disclosure include but are not limited to porous membranes, ceramics, fibers, glass, fiberglass, polypropylene, polyethylene terephthalate, cellulose, plastics, meshes, foams, sponges, high-efficiency particulate air (HEPA) filters, combinations thereof, and the like.

It is contemplated that the combination of an activated carbon filter with a second filter which does not include activated carbon may allow the size and/or complexity of the filtration system to be reduced, the life of the filtration system to be extended, or combinations thereof, without wishing to be bound by theory.

In some embodiments, the filtration system of the present disclosure may be used in a single-pass system. In other embodiments, the filtration system of the present disclosure may be used in a multi-pass system, wherein a gas stream is continually cycled through the filtration system. The filtration system of the present disclosure may be portable or fixed, according to some embodiments.

In some embodiments, there is provided a method of filtering viral particles from a gas stream which contains viral particles. The method may, in some embodiments, include contacting the gas stream with a filtration system as described herein, such as a filtration system which includes a first filter including activated carbon impregnated with an additive which includes iron, copper, zinc, cerium, silver, a quaternary ammonium compound, or combinations thereof, and a second filter. In some embodiments of the disclosed method, the second filter does not include activated carbon, and may include any of the non-activated carbon filter materials and designs described herein.

In some embodiments, contacting the filtration system with the gas stream results in at least about 95% reduction in viral particles, such as at least about 95%, at least about 96%, at least about 97%, at least about 98%, at least about 99%, about 100%, or any range or value contained therein.

The systems and methods described herein may be of particular use in filtration applications in confined spaces, including but not limited to automobiles, airplanes, trains, subways, water vessels such as boats, ships, and submarines, spacecraft, construction zones, and the like. Other possible uses for the systems and methods of the present disclosure include but are not limited to hospitals, schools, airports, train stations, museums, restaurants, restrooms including portable restrooms, home air purifiers, HVAC systems, and other locations or areas where there may be a need to filter viral particles from air. The embodiments described herein may be combined in any fashion to form new embodiments.

### EMBODIMENTS

In one embodiment, an activated carbon material is used for filtering unwanted contaminants from air.

In another embodiment, the unwanted particulates are viral particles.

In another embodiment, the unwanted particles are bacterial particles.

In another embodiment, the unwanted particles are germs or other infectious agents.

In another embodiment, the activated carbon is impregnated with an additive.

In another embodiment, the activated carbon is impregnated with a metal additive or a metal-containing additive.

In another embodiment, the activated carbon is impregnated with a quaternary ammonium compound additive.

In one embodiment, a composition for filtering viral particles from air, comprises activated carbon and a metal additive, wherein the metal may be any of iron, cobalt, cerium, nickel, copper, zinc, or silver, or oxides thereof, or a quaternary ammonium compound.

In another embodiment, the activated carbon may be sourced from any of bagasse, bamboo, coconut husks, peat, wood such as hardwood and softwood sources, for example in the form of sawdust and scrap, lignite, coal and coal tar, petroleum pitch, asphalt and bitumen, corn stalks and husks, wheat straw, spent grains, rice hulls and husks, nutshells, and combinations thereof.

In another embodiment, the additive is iron, iron oxide, or iron oxyhydroxide.

In another embodiment, the additive is copper or copper oxide.

In another embodiment, the additive is cuprous oxide or cupric oxide.

In another embodiment, the additive is cupric oxide.

In another embodiment, the additive is cuprous oxide.

In another embodiment, the additive is zinc or zinc oxide.

In another embodiment, the additive is silver or silver oxide.

In another embodiment, the additive is a quatemary ammonium compound.

In another embodiment, the additive is benzalkonium chloride.

In another embodiment, the activated carbon comprises about 0.1 wt. % to about 10 wt. % of the additive.

In another embodiment, the activated carbon comprises about 1 wt. % to about 5 wt. % of the additive.

In another embodiment, the activated carbon comprises about 2 wt. % to about 7 wt. % of the additive.

In another embodiment, the activated carbon comprises about 5 wt. % to about 10 wt. % of the additive.

In another embodiment, the activated carbon comprises about 0.2 wt. % to about 1 wt. % of the additive.

In another embodiment, the composition provides at least a 95% reduction in viral particles when contacted with a gas stream containing viral particles.

In one embodiment, a method of filtering viral particles from air, the method comprising using an activated carbon impregnated with an additive of any of iron, copper, zinc, or silver, or oxides thereof.

In another embodiment, the impregnated activated carbon is contained within a filter and is used in combination with a second filter which does not include activated carbon.

In another embodiment, the impregnated activated carbon is housed within a filter system.

In another embodiment, the filter system may be a cabin air filter, a home air filter, an HVAC filter, or the like.

In another embodiment, the filter system is wearable.

In another embodiment, the filter system is a face mask.

### EXAMPLES

Although anti-viral activated carbon filtration products have been described herein in considerable detail with reference to certain preferred embodiments thereof, other versions are possible. Therefore, the spirit and scope of the appended claims should not be limited to the description and the preferred versions contained within this specification. Various aspects of the anti-viral activated carbon filtration products will be illustrated with reference to the following non-limiting examples.

### Example 1

Activated carbon may be formed from any of a number of starting sources. Activated carbon may be further treated with additional procedures or additives to induce desired properties, or used in an untreated form. In this example, the activated carbon used is a coal-based, 35×60 virgin activated carbon.

### Example 2

In this example, silver nitrate was added to activated carbon followed by heat treatment at about 425° C. under nitrogen. This process produced an activated carbon impregnated with silver at approximately 4% by weight.

### Example 3

Activated carbon was treated with standard copper carbonate-ammonia impregnation, followed by drying in air to about 200° C. Resulting copper content was about 8% by weight.

### Example 4

Activated carbon was subjected to standard zinc oxide-ammonia impregnation, followed by drying in air to about 200° C. Resulting zinc content was about 8% by weight.

### Example 5

Activated carbon was impregnated with iron salts that were first converted with aqueous sodium hydroxide, followed by air drying for at least ten hours at about 105° C. Resulting iron content was about 2% by weight.

### Example 6

Activated carbon was impregnated a quaternary ammonium compound (benzalkonium chloride). The resulting benzalkonium chloride content was about 0.5% by weight.

Anti-Viral Testing

Activated carbon was evaluated for single-pass efficacy against aerosolized MS2, a single-stranded viral RNA bacteriophage that may be used as a representative surrogate for influenza and other viruses. The efficacy of several activated carbon compositions was assessed via an upstream and downstream sampling method in a custom stainless steel bioaerosol challenge system. Comparison of the upstream and downstream samples yielded single-pass efficacy in terms of the percent and LOG reduction of the bioaerosol challenge.

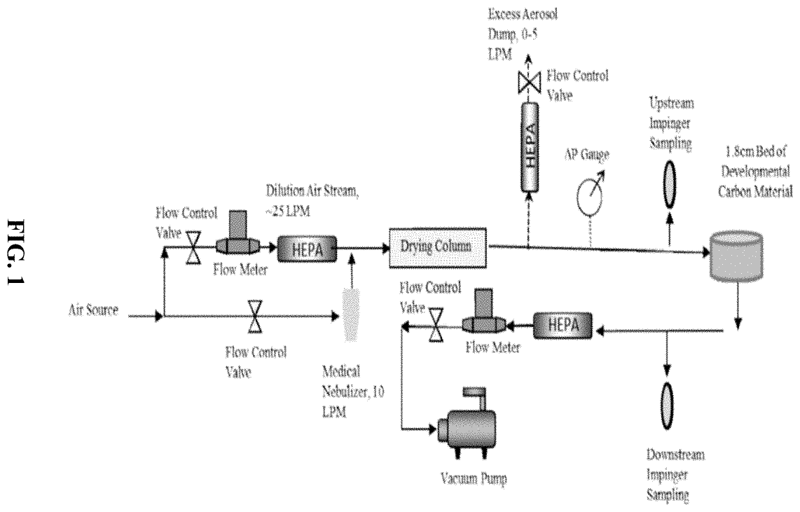

Testing was conducted to evaluate the single-pass and reduction capabilities of five activated carbon compositions containing additives and one activated carbon control sample in a 0.7 in (1.8 cm) bed with a diameter of 1.5 in (3.8 cm). A total of 18 single-pass trials were conducted on six activated carbon materials, including one control and five additional compositions that included various additives. The bioaerosol testing system was assembled using sanitary stainless-steel fittings, AGI-30 impingers, a medical nebulizer, HEPA filters, and a vacuum pump. The flow diagram of this system is shown in FIG. 1.

Test bioaerosols were disseminated using a medical nebulizer driven by HEPA filtered house air supply at 30 PSI. A pressure regulator allowed for control of disseminated particle size, use rate, and sheer force generated within the nebulizer.

A pair of AGI-30 impingers were used for bioaerosol sample collection for all fifteen single pass trials conducted. The impingers were filled with 20 mL of Phosphate Buffered Saline (PBS) solution for collection of the bioaerosol. The impingers were then serially diluted and plated for direct enumeration of colony forming units (cfu).

The impinger flow vacuum source was maintained using a valved Emerson 1.3 hp rotary vane pump (Emerson Electric, St. Louis, Mo.) equipped with a 0-30 inches Hg vacuum gauge (WIKA Instruments, Lawrenceville, Ga.). The pump was operated at a negative pressure of 18 inches of Hg during all characterization and control valves and flow meters were placed in line with impingers to control the sample flow rate of the impingers. The AGI-30 impingers sample at a flow rate of 12.5 LPM.

Lipid-enveloped viruses are the least resistant microorganisms to germicidal chemicals. It is presumed that this susceptibility is similar for other chemical, physical, and catalytic methods of destruction. MS2 is a non-enveloped virus, which makes it more resistant to disinfection than lipid viruses, and therefore should represent a “worst case scenario” when compared to lipid-enveloped viruses such as SARS-CoV-2. It is thus expected that efficacy against MS2 suggests efficacy against other viruses such as influenza and SARS-CoV-2.

Pure strain viral seed stock and host bacterium stock were obtained. Host bacterium stock was grown in a similar fashion to vegetative cells in an appropriate liquid media. The liquid media was infected during the logarithmic growth cycle with the specific bacteriophage. After an appropriate incubation time, the cells were lysed and the cellular debris separated by centrifugation. MS2 stock yields were greater than 1×10¹¹ plaque forming units per milliliter (pfu/mL) for use in the medical nebulizer.

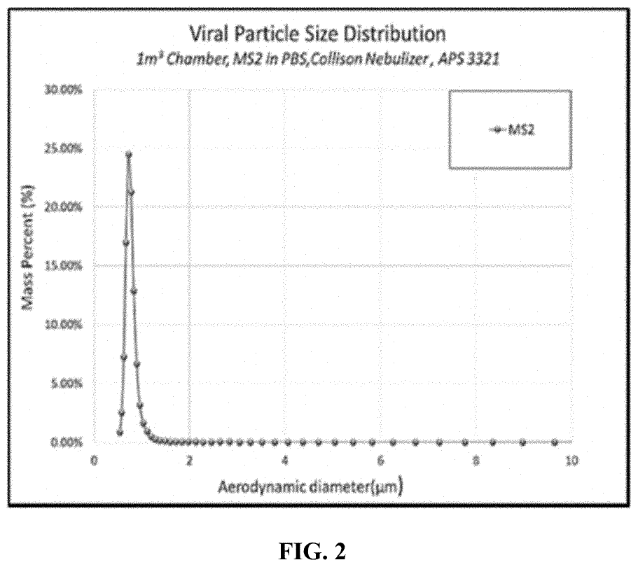

Aerosol particle size distributors were sampled and measured with TSI Aerodynamic Particle Sizer (APS). The APS has a dynamic measurement range of 0.5 to 20 μm and was used to determine particle size and initial concentrations at the onset of testing. Particle size data was collected for the bioaerosol challenge. An average MS2 bacteriophage particle size distribution can be found in FIG. 2.

The activated carbon material in granular form was poured into a custom holder with a 1.8 cm bed that was integrated into the custom single pass system. The granular bed measured 1.8 cm by 3.8 cm. Upstream and downstream sampling was performed using AGI-30 impingers to sample upstream and downstream, which sample at 12.5 LPM. All test materials were pre-humidified using deionized water at 60% relative humidity for fifteen (15) minutes prior to testing.

All testing was conducted at a total flow rate of 20.5 LPM through the test system with a face velocity of 30 cm/sec with a contact time of about 60 milliseconds. A HEPA filtered excess air dump was integrated into the system in order to remove excess air from the system. For testing, dilution air was turned on at a flow rate of 30 liters per minute (LPM) at the beginning of each trial. A HEPA filtered excess air dump was integrated into the system for excess dilution air to exit the system. Then the main system vacuum pump was turned on downstream of the activated carbon, operating at 8 LPM. This combined with the 12.5 LPM being pulled by the downstream impinger equals a total flow rate of 20.5 LPM. The nebulizer was then turned on and operated at a pressure of 30 psi. Upstream and downstream impingers were turned on and sampled for three minutes in order to assure adequate sample collection in the downstream impinger.

After testing each material, HEPA filtered house air at 40 LPM was flowed through the system for about 10 minutes in order to ensure that the system had no remaining bioaerosols. Once this system purge was completed, a fresh 1.8 cm bed of the next activated carbon material was loaded into the holder for the next test. Filter samples were replaced after each individual trial was conducted.

Impinger and stock biological cultures were serially diluted and plated in triplicate using a small drop assay technique onto tryptic soy agar plates. The plated cultures were incubated for 24 hours and enumerated and recorded.

Following each trial, the nebulizer was cleaned and filled with 35% hydrogen peroxide. The peroxide was nebulized for approximately fifteen minutes while 25 LPM of HEPA filtered air was run through the system. The nebulizer was then turned off and the dilution air continued to run through the system for an additional fifteen minutes in order to ensure that all hydrogen peroxide was removed from the system before beginning the next trial.

Efficacy Results

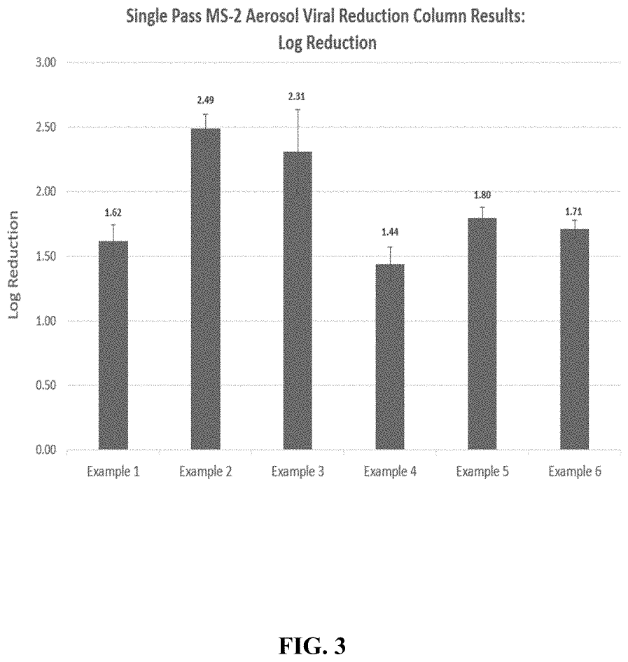

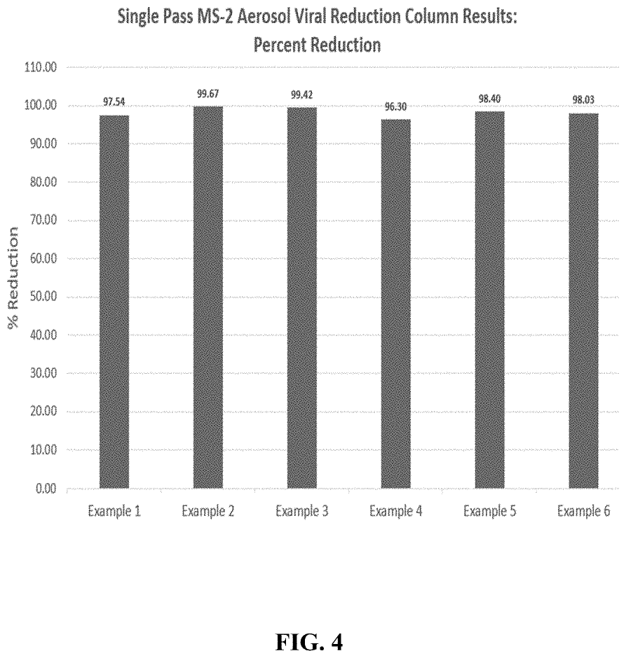

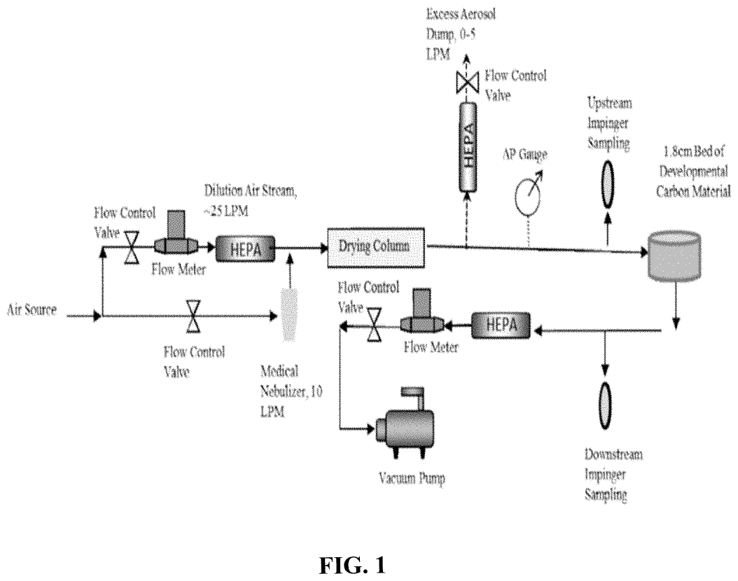

Single pass results for MS2 bacteriophage testing showed average LOG reduction values between 1.44 and 2.49 for all six materials, with average values and standard deviations shown in FIG. 3. The reduction in terms of percentage for each of the six materials is shown in FIG. 4. The control material, a coal-based, virgin activated carbon (Example 1), showed a LOG reduction of 1.62 LOG (97.545). Example 2 showed the highest LOG reduction of 2.49 LOG (99.67%), only slightly higher than Example 3 with a LOG reduction of 2.31 LOG (99.42%). Example 4 showed the lowest LOG reduction of 1.44 LOG (96.30%). Example 5 showed a LOG reduction of 1.80 LOG (98.40%). Example 6 showed a LOG reduction of 1.71 LOG (98.03%). All materials including the control showed a greater than 1 LOG reduction (90%), which suggests the activated carbon materials are all effective at removing aerosolized MS2 bacteriophage.

### Evaluation of Efficacy Against MS2 Bacteriophage—Control and Example Materials

An untreated control material (Example 1) and treated compositions Examples 2-6 were evaluated for efficacy against MS2 bacteriophage. In this test, the activated carbon samples were placed in a bed and contacted with a stream of air containing viral particles. The resulting viral particle concentration in air after contacting the compositions was evaluated. Results are illustrated in TABLE 1. Each Example was tested three times, indicated by T1, T2, and T3 in TABLE 1.

TABLE 1

| | Percent | LOG |
|---|---|---|
| Trial | Reduction | Reduction |
| Example 1 T1 | 98.00% | 1.70 |
| Example 1 T2 | 96.65% | 1.48 |
| Example 1 T3 | 97.97% | 1.69 |
| Example 1 Average | 97.54% | 1.62 |
| Ex. 1 Standard Deviation | 0.77% | 0.13 |
| Example 2 T1 | 99.57% | 2.37 |
| Example 2 T2 | 99.73% | 2.57 |
| Example 2 T3 | 99.71% | 2.54 |
| Example 2 Average | 99.67% | 2.49 |
| Ex. 2 Standard Deviation | 0.09% | 0.11 |
| Example 3 T1 | 99.78% | 2.65 |
| Example 3 T2 | 99.48% | 2.28 |
| Example 3 T3 | 99.00% | 2.00 |
| Example 3 Average | 99.42% | 2.31 |
| Ex. 3 Standard Deviation | 0.39% | 0.32 |
| Example 4 T1 | 96.00% | 1.40 |
| Example 4 T2 | 97.43% | 1.59 |
| Example 4 T3 | 95.48% | 1.34 |
| Example 4 Average | 96.30% | 1.44 |
| Ex. 4 Standard Deviation | 1.01% | 0.13 |
| Example 5 T1 | 98.19% | 1.74 |
| Example 5 T2 | 98.28% | 1.76 |
| Example 5 T3 | 98.72% | 1.89 |
| Example 5 Average | 98.40% | 1.80 |
| Ex. 5 Standard Deviation | 0.28% | 0.08 |
| Example 6 T1 | 98.32% | 1.78 |
| Example 6 T2 | 98.09% | 1.72 |
| Example 6 T3 | 97.69% | 1.64 |
| Example 6 Average | 98.03% | 1.71 |
| Ex. 6 Standard Deviation | 0.32% | 0.07 |

In the above detailed description, reference is made to the accompanying drawings, which form a part hereof. In the drawings, similar symbols typically identify similar components, unless context dictates otherwise. The illustrative embodiments described in the detailed description, drawings, and claims are not meant to be limiting. Other embodiments may be used, and other changes may be made, without departing from the spirit or scope of the subject matter presented herein. It will be readily understood that the aspects of the present disclosure, as generally described herein, and illustrated in the Figures, can be arranged, substituted, combined, separated, and designed in a wide variety of different configurations, all of which are explicitly contemplated herein.

The present disclosure is not to be limited in terms of the particular embodiments described in this application, which are intended as illustrations of various aspects. Many modifications and variations can be made without departing from its spirit and scope, as will be apparent to those skilled in the art. Functionally equivalent methods and apparatuses within the scope of the disclosure, in addition to those enumerated herein, will be apparent to those skilled in the art from the foregoing descriptions. Such modifications and variations are intended to fall within the scope of the appended claims. The present disclosure is to be limited only by the terms of the appended claims, along with the full scope of equivalents to which such claims are entitled. It is to be understood that this disclosure is not limited to particular methods, reagents, compounds, compositions or biological systems, which can, of course, vary. It is also to be understood that the terminology used herein is for the purpose of describing particular embodiments only, and is not intended to be limiting.

With respect to the use of substantially any plural and/or singular terms herein, those having skill in the art can translate from the plural to the singular and/or from the singular to the plural as is appropriate to the context and/or application. The various singular/plural permutations may be expressly set forth herein for sake of clarity.

It will be understood by those within the art that, in general, terms used herein, and especially in the appended claims (for example, bodies of the appended claims) are generally intended as “open” terms (for example, the term “including” should be interpreted as “including but not limited to,” the term “having” should be interpreted as “having at least,” the term “includes” should be interpreted as “includes but is not limited to,” et cetera). While various compositions, methods, and devices are described in terms of “comprising” various components or steps (interpreted as meaning “including, but not limited to”), the compositions, methods, and devices can also “consist essentially of” or “consist of” the various components and steps, and such terminology should be interpreted as defining essentially closed-member groups. It will be further understood by those within the art that if a specific number of an introduced claim recitation is intended, such an intent will be explicitly recited in the claim, and in the absence of such recitation no such intent is present.

For example, as an aid to understanding, the following appended claims may contain usage of the introductory phrases “at least one” and “one or more” to introduce claim recitations. However, the use of such phrases should not be construed to imply that the introduction of a claim recitation by the indefinite articles “a” or “an” limits any particular claim containing such introduced claim recitation to embodiments containing only one such recitation, even when the same claim includes the introductory phrases “one or more” or “at least one” and indefinite articles such as “a” or “an” (for example, “a” and/or “an” should be interpreted to mean “at least one” or “one or more”); the same holds true for the use of definite articles used to introduce claim recitations.

In addition, even if a specific number of an introduced claim recitation is explicitly recited, those skilled in the art will recognize that such recitation should be interpreted to mean at least the recited number (for example, the bare recitation of “two recitations,” without other modifiers, means at least two recitations, or two or more recitations). Furthermore, in those instances where a convention analogous to “at least one of A, B, and C, et cetera” is used, in general such a construction is intended in the sense one having skill in the art would understand the convention (for example, “a system having at least one of A, B, and C” would include but not be limited to systems that have A alone, B alone, C alone, A and B together, A and C together, B and C together, and/or A, B, and C together, et cetera). In those instances where a convention analogous to “at least one of A, B, or C, et cetera” is used, in general such a construction is intended in the sense one having skill in the art would understand the convention (for example, “a system having at least one of A, B, or C” would include but not be limited to systems that have A alone, B alone, C alone, A and B together, A and C together, B and C together, and/or A, B, and C together, et cetera). It will be further understood by those within the art that virtually any disjunctive word and/or phrase presenting two or more alternative terms, whether in the description, claims, or drawings, should be understood to contemplate the possibilities of including one of the terms, either of the terms, or both terms. For example, the phrase “A or B” will be understood to include the possibilities of “A” or “B” or “A and B.”

In addition, where features or aspects of the disclosure are described in terms of Markush groups, those skilled in the art will recognize that the disclosure is also thereby described in terms of any individual member or subgroup of members of the Markush group.

As will be understood by one skilled in the art, for any and all purposes, such as in terms of providing a written description, all ranges disclosed herein also encompass any and all possible subranges and combinations of subranges thereof. Any listed range can be easily recognized as sufficiently describing and enabling the same range being broken down into at least equal halves, thirds, quarters, fifths, tenths, et cetera. As a non-limiting example, each range discussed herein can be readily broken down into a lower third, middle third and upper third, et cetera. As will also be understood by one skilled in the art all language such as “up to,” “at least,” and the like include the number recited and refer to ranges that can be subsequently broken down into subranges as discussed above. Finally, as will be understood by one skilled in the art, a range includes each individual member. Thus, for example, a group having 1-3 layers refers to groups having 1, 2, or 3 layers. Similarly, a group having 1-5 layers refers to groups having 1, 2, 3, 4, or 5 layers, and so forth.

Various of the above-disclosed and other features and functions, or alternatives thereof, may be combined into many other different systems or applications. Various presently unforeseen or unanticipated alternatives, modifications, variations or improvements therein may be subsequently made by those skilled in the art, each of which is also intended to be encompassed by the disclosed embodiments.

What is claimed is:

1. A filtration system for filtering viral particles from a gas stream, comprising:
a first filter comprising activated carbon impregnated with an additive comprising iron, cobalt, cerium, nickel, copper, zinc, silver, a quaternary ammonium compound, or combinations thereof, and
a second filter which does not comprise activated carbon.

2. The filtration system of claim 1, wherein the activated carbon is formed from at least one of bagasse, bamboo, coconut husks, peat, wood such as hardwood and softwood sources in the form of sawdust and scrap, lignite, coal, coal tar, petroleum pitch, asphalt and bitumen, corn stalks and husks, wheat straw, spent grains, rice hulls and husks, nutshells, or combinations thereof.

3. The filtration system of claim 1, wherein the activated carbon is granular.

4. The filtration system of claim 1, wherein the additive comprises elemental iron, iron oxide, iron oxyhydroxide, elemental copper, cuprous oxide, cupric oxide, elemental zinc, zinc oxide, elemental silver, silver oxide, silver nitrate, silver sulfate, silver phosphate, benzalkonium chloride, or combinations thereof.

5. The filtration system of claim 1, wherein the activated carbon comprises about 0.1 wt. % to about 10 wt. % of the additive.

6. The filtration system of claim 1, wherein the activated carbon comprises about 0.1 wt. % to about 2 wt. % of the additive.

7. The filtration system of claim 1, wherein the activated carbon comprises about 1 wt. % to about 5 wt. % of the additive.

8. The filtration system of claim 1, wherein the activated carbon comprises about 5 wt. % to about 10 wt. % of the additive.

9. The filtration system of claim 1, wherein the second filter comprises ceramic, porous membranes, fibers, glass, fiberglass, polypropylene, polyethylene terephthalate, cellulose, plastic, mesh, foam, sponge, high-efficiency particle air filter, or combinations thereof.

10. The filtration system of claim 1, wherein the filtration system is contained within a housing, a wearable device, or combinations thereof.

11. The filtration system of claim 1, wherein contacting the filtration system with the gas stream results in at least about 95% reduction in viral particles.

12. A method of filtering viral particles from a gas stream that contains viral particles, comprising:
contacting the gas stream with a filtration system comprising:
a first filter comprising activated carbon impregnated with an additive comprising iron, copper, zinc, cerium, silver, a quaternary ammonium compound, or combinations thereof, and
a second filter.

13. The method of claim 12, wherein the activated carbon is formed from at least one of bagasse, bamboo, coconut husks, peat, wood such as hardwood and softwood sources in the form of sawdust and scrap, lignite, coal, coal tar, petroleum pitch, asphalt and bitumen, corn stalks and husks, wheat straw, spent grains, rice hulls and husks, nutshells, or combinations thereof.

14. The method of claim 12, wherein the activated carbon is granular.

15. The method of claim 12, wherein the activated carbon comprises about 0.1 wt. % to about 10 wt. % of the additive.

16. The method of claim 12, wherein the second filter does not comprise activated carbon.

17. The method of claim 12, wherein the second filter comprises ceramic, porous membranes, fibers, glass, fiberglass, polypropylene, polyethylene terephthalate, cellulose, plastic, mesh, foam, sponge, high-efficiency particle air filter, or combinations thereof.

18. The method of claim 12, wherein the filtration system is contained within a housing, a wearable device, or combinations thereof.

19. The method of claim 12, wherein contacting the gas stream with the filtration system results in at least about 95% reduction in viral particles.

20. The method of claim 12, wherein contacting the gas stream with the filtration system results in at least about 98% reduction in viral particles.
